(19) United States
(12) Patent Application Publication
(10) Pub. No.: US 2016/0146838 A1
(43) Pub. Date: May 26, 2016

(54) SYSTEM AND METHOD FOR EBOLA DETECTION

(71) Applicant: Jean Ronald Brisard, Englewood, NJ (US)

(72) Inventor: Jean Ronald Brisard, Englewood, NJ (US)

(21) Appl. No.: 14/548,314

(22) Filed: Nov. 20, 2014

(51) Int. Cl. G01N 33/72

(52) U.S. Cl. CPC G01N 33/728; G01N 2800/26; G01N 2333/43504

(57) ABSTRACT

A system and method for determining the level of bilirubin in bodily fluids based on the use protein that binds with bilirubin and measuring the level of bioluminescence produced and the use of measurements over time as a means of predicting potential infection and in particular possible Ebola infection.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### FIELD

The present application relates to detection of disease and particularly to the detection of Ebola using fluorescence.

### BACKGROUND

In the course of human history, humankind has been affected with countless plagues and other medical calamities/contagious diseases caused by bacteria, viruses and sometimes parasites.

A great number of these dreadful diseases are caused by relevant pathogens (viruses, bacteria, or fungi) that may be spread through coughing, sneezing, raising of dust, spraying of liquids, or similar activities likely to generate aerosol particles or droplets. Others are spread via contact with contaminated human body fluids of a carrier, which may or may not be symptomatic themselves. These body fluids usually include blood, urine, fecal matter, vomit, sweat and even saliva or mucus.

The current Ebola epidemic spreading across the globe illustrates the challenge posed by controlling and managing the spread of a highly infectious and deadly disease. Unfortunately, detecting Ebola is not a quick process. In a hospital it can take up to five days for test results to be confirmed. Once a blood sample is taken, it must be tested in a lab. Exacerbating the problem is that those infected with Ebola show no symptoms or outward signs of infection while the virus incubates, which can take up to 21 days.

According to the World Health Organization, patients are only contagious as soon as they start showing symptoms. Unfortunately, by that point, they may have had already come into close contact with numerous other people. As a result, this long incubation period greatly increases the risk of spreading the disease.

Another important issue regarding the Ebola crisis is the risk of contamination of close family members and healthcare workers who are exposed to the infected body fluids of the sick patient. Ebola, like many other contagious diseases, causes profuse vomiting and diarrhea. These excreta, as well as the urine contain a very high viral load and are therefore very infectious. It is therefore imperative that those who are treating and handling Ebola patients wear protective clothing and follow strict infection control protocols in order to avoid being infected with the virus. All infected excreta and body fluids and soiled materials must also be handled with extreme care and be disposed of properly. Additionally, all contaminated area must also be thoroughly cleaned and disinfected.

There are a number of Ebola detection kits currently in use with the most common types using Polymerase Chain Reaction (PCR) to detect the presence of the virus. Unfortunately, these test take anywhere from 5-8 hours, with only a typical accuracy of 90%, and requires a highly secured and specialized laboratory where the infected patient's body fluids are examined.

However, the biggest limitation of these testing kits is that they will not reveal or confirm the presence of the Ebola virus during the incubation period, which as previously mentioned can last up to 21 days.

Therefore, there continues to be a need for an early detection system, which can detect the presence of Ebola during the incubation period.

### SUMMARY

In order to overcome the deficiencies in the prior art, systems and methods are described herein.

One aspect of the claimed invention involves an Ebola detection method comprising adding a protein to a sample of bodily fluid that combines bilirubin in the sample, and is fluorescent when combined, measuring the fluorescence in the sample in order to determine the level of bilirubin, and determining based upon the relative level bilirubin in the sample whether or not the bodily fluids show indications of Ebola infection.

Another aspect involves a bilirubin level detection system comprising a sample chamber; a light source configured to transmit light into the sample chamber at a frequency appropriate to produce a bioluminescent effect in a sample when placed in the chamber; and accompanying electronic circuitry configured to process, measure, and display the level of fluorescence in the sample.

These and other aspects described herein present in the claims result in features and/or can provide advantages over current technology.

The advantages and features described herein are a few of the many advantages and features available from representative embodiments and are presented only to assist in understanding the invention. It should be understood that they are not to be considered limitations on the invention as defined by the claims, or limitations on equivalents to the claims. For instance, some of these advantages or features are mutually exclusive or contradictory, in that they cannot be simultaneously present in a single embodiment. Similarly, some advantages are applicable to one aspect of the invention, and inapplicable to others. Thus, the elaborated features and advantages should not be considered dispositive in determining equivalence. Additional features and advantages of the invention will become apparent in the following description, from the drawings, and from the claims.

### BRIEF DESCRIPTION OF THE DRAWINGS

This disclosure is further described in the detailed description that follows, with reference to the drawings, in which:

FIG. 1A shows, in simplified form, a method of determining the concentration of bilirubin using reagent wells;

FIG. 1B shows, in simplified form, the resultant fluorescence level intensity measure for each well; and

FIG. 2 shows, in simplified form, an apparatus for measuring fluorescence.

### DETAILED DESCRIPTION

The instant devices and approach provide an Ebola detection system based upon the principle that elevated bilirubin levels in bodily fluids may be precursor to Ebola infection and that the level of bilirubin in the bodily fluid can be measured by combining the bodily fluid with a special protein that fluoresces when bond to the bilirubin in the bodily fluid.

Bilirubin is a natural byproduct generated by the breakdown of red blood cells. As the liver processes the bilirubin, it is transformed and processed as bile in the gallbladder. Some of the bilirubin is re-absorbed into the blood and the rest excreted in feces or urine.

One of the first signs of liver disease is jaundice, or a yellowing of the skin and eyes. Jaundice is caused by an accumulation of too much bilirubin in the blood.

Fever is currently the first sign known indicator of possible Ebola infection. When a person is suspected of being exposed to the Ebola virus, he/she is placed under observation or quarantine, while a daily recording of the temperature is taken.

Because fever is the manifestation of the fact that abnormal metabolic processes are taking place in the body, it is anticipated that these abnormal metabolic changes may be occurring for days before the fever manifests itself. While the fever make take days to show up, it is anticipated that one of the early metabolic changes that occurs when someone has been infected is a spike in the level bilirubin in their bodily fluids, specifically blood, feces, and urine but it may also be detectable in vomit, sweat, saliva and/or mucus as well.

The early detection of an increased bilirubin level, while not specific to Ebola, can serve as a strong indicator that the deadly viral infection may be present, especially in a patient already being under observation. As such, a spike in bilirubin would warrant the immediate activation of more stringent quarantine procedures and more specific testing for the Ebola virus in the patient. To this end, a system for and method of determining bilirubin levels bodily fluids will now be described.

In a recent scientific paper by Kumagai, Akiko, et al. “A bilirubin-inducible fluorescent protein from eel muscle.” Cell 153.7 (2013): 1602-1611, which is hereby incorporated by reference, Kumagai, et al., have identified a specific protein from the muscle of a Japanese eel, UnaG, that not only has bioluminescent properties but that research shows has a very strong affinity for binding specificity to un-conjugated bilirubin. Moreover, this binding also causes the UnaG protein to become highly bioluminescent, when exposed to a specific type of ultra-violet light.

Using UnaG, or other similar reagent proteins that have the ability to bond with bilirubin, the level of bilirubin can be determined in a bodily fluid test (e.g. a urine or blood test). In a bodily fluid test, a sample of the bodily fluid is mixed with the reagent solution containing the fluorescent protein. The bilirubin in the bodily fluid is combined to the fluorescent protein and the bilirubin level is determined based upon the amount of bioluminescence produced.

The affinity and specificity of the binding of the UnaG and the bilirubin has been determined at a Kd value of 98 pM, where Kd, the Dissociation constant is measured in pico Molar units. Kd essentially is a measure of how many molecules of Bilirubin are bonded to the Protein and how tight the bond is.

For example, suppose we have the reversible reactions at equilibrium

B+P<====>B−P (EQ 1)

where B is free Bilirubin, P is ligand (protein fragment, in this case UnaG), and B-P is the Bilirubin-UnaG complex which is held together by intermolecular forces, not covalent forces. Then, Kd, is given by the equation

[B]_{eq}[P]_{eq}/[B−P]_{eq} (EQ 2)

which is the ratio of the concentrations of free B and free P compared to the bound BP complex and eq is at equilibrium. The unit of Kd is molarity and the lower the Kd, the tighter the binding, and the higher the Kd, the looser the binding.

Since the Kd of the Bilirubin-UnaG is known, the Bilirubin level can be derived by using the above formula

Kd=[B]_{eq}[P]_{eq}/[B−P]_{eq}. (EQ 3)

Since there is a high affinity between bilirubin and UnaG (low Kd of 98 pM), it is expected that in a solution with a high concentration of UnaG, all the Bilirubin molecules added will be essentially 100% bound to the fluorescent protein. As a result a standard technique for determining concentration is to use reagent wells, other techniques include measuring fluorescence against pre-calibrated samples.

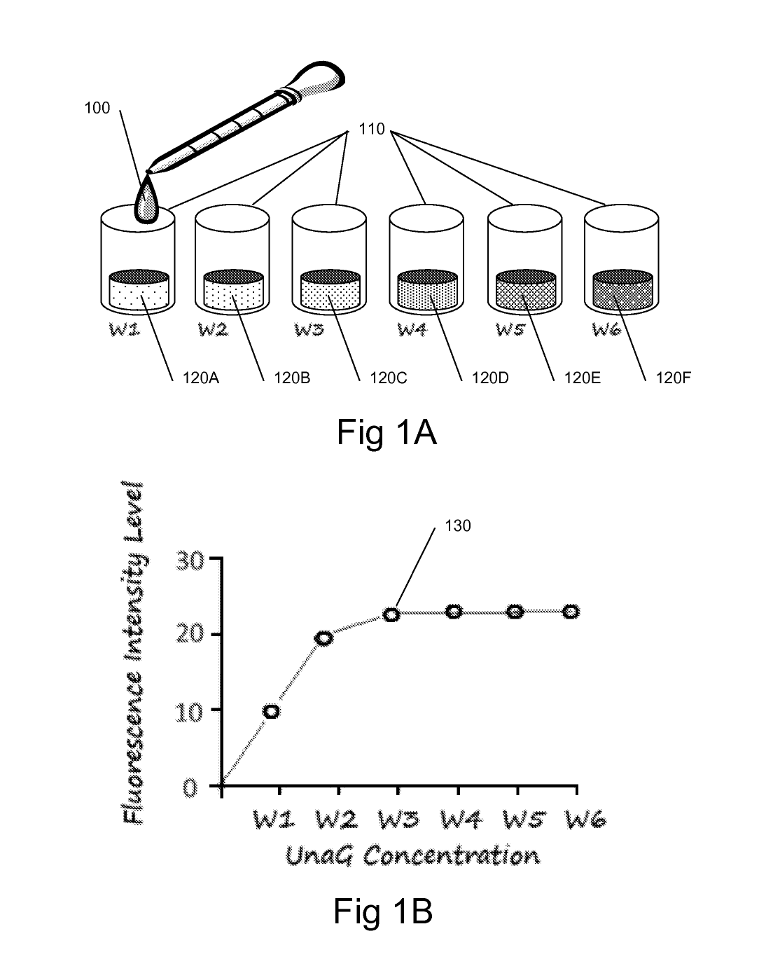

FIG. 1A shows, in simplified form, a method of determining the concentration of bilirubin using reagent wells. In FIG. 1, a few drops of the bodily fluid 100 sample is added to a series of reagent wells 110, each containing a different concentration of the reagent protein 120A, 120B, 120C, 120D, 120E, 120F. The reagent wells are labeled as W1-W6, with W1 having the least concentration and W6 having the highest concentration. FIG. 1B shows, in simplified form, the resultant fluorescence level intensity measure for each well, W1-W6. In FIG. 1B, we see that the bilirubin molecules will saturate all the lower concentration wells, W1 and W2, except where the well concentration of the reagent protein is higher than the bilirubin concentration 130. This well, W3, is referred to as the Peak-Well. The fluorescence levels of all wells, W4-W6, past the Peak-Well will be the same, since all the bilirubin in the sample is already 100% bound with any extra ligand (protein). As a result, the higher concentration will not find any more bilirubin molecules to bind with or produce any significant increase in fluorescence intensity.

A representative protocol for testing urine (or another bodily fluid), using a device comprising one or more wells as described in FIG. 1, involves: using a clean plastic cup or container to collect the first urination of the day (or the urination after first urination); using an eye-drop to transfer 3 to 5 drops of urine to each well in the test device; close each well tightly and gently shake the test device for a 3 to 5 seconds or other pre-determined time increment; letting the test device sit at room temperature for at least 6-10 minutes, to allow bilirubin to combine with the protein or other pre-determined time increment in order to allow equilibrium (or near equilibrium such that there is no discernible difference based upon the measurement system accuracy) to be achieved, which may be shortened with the use of an accelerating factor or accelerating process such as heating (or cooling), but not more than 1 hour or other predetermined time increment (e.g. for instance after approximately 2 hours a form of oxidation, azo-pigmentation, can transform bilirubin from un-conjugated to conjugated) associated with stability of the reaction, which may be lengthened through the use of a stabilizing factor or stabilizing process such as cooling (or heating); determining which concentration produced the peak illumination; repeating the process for subsequent urinations (or based upon pre-determined intervals, such as the first urination of each day), and comparing the differential levels of bilirubin determined to see if it has increased beyond a pre-determined threshold and if so issuing a warning that the patient may have an Ebola infection or other potential process associated with a spike in bilirubin levels.

Alternatively, once the relationship between fluorescence and bilirubin concentration has been established then, using a known quantity of the reagent agent, the fluorescence level measured can used to determine the bilirubin concentration in a bodily fluid, assuming the reagent agent has been saturated and systems for and methods of using relationship between fluorescence and bilirubin concentration will now be discussed.

The measurement of fluorescence is well known in the art and referred to as fluorescence spectroscopy (also known as fluorometry or spectrofluorometry) and is a type of electromagnetic spectroscopy, which analyzes fluorescence from a sample. It involves using a beam of light, usually ultraviolet light, that excites the electrons in molecules of certain compounds and causes them to emit light; typically, but not necessarily, visible light. A complementary technique is absorption spectroscopy. Devices that measure fluorescence are called, fluorometers or fluorimeters.

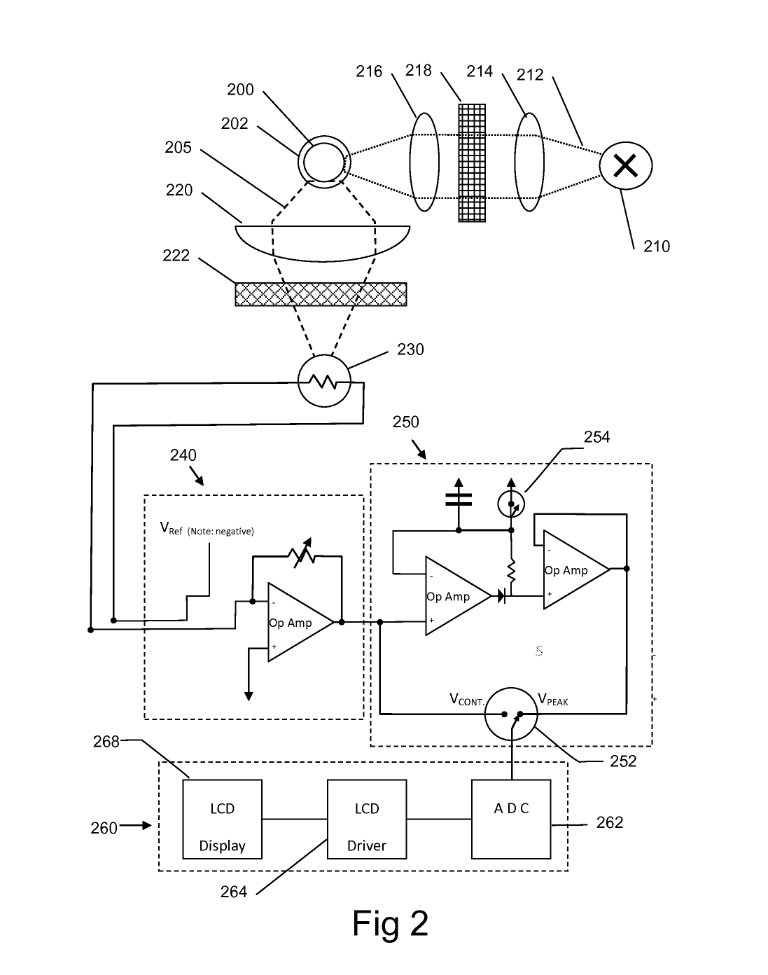

As an aid in understanding, FIG. 2 show, in simplified form, an apparatus for measuring fluorescence. FIG. 2 shows a sample 200 in a sample chamber 202, receiving light 212 from a source 210 after it passes through source lens 214, 218, deigned to focus the light, and source filter 216, designed to produce light of a 202 specific wavelength. A portion of the light 212 received by the sample is absorbed and then re-emitted as fluorescent light 205. As the light 205 travels in all directions, a receiving lens 220 is typically used to focus the light onto the receiver 230 after it passes through a receiver filter 222, designed to filter out all light except the light that corresponds to a specific frequency of the emitted light.

In practice the source 210 and the receiver are typically placed 90 degrees to one another in order to minimize the amount of reflected light 212 from reaching the receiver 230 but other configurations are possible. Additionally, it should be noted that depending on the source 210 that is used, it is often possible to eliminate the source lens 214, 218 and source filter 218, for example if laser, with a very narrow beam and right frequency is used since. In a similar vain, the receiving filter, which is represented as a discrete component could be performed as a filtering algorithm performed in software post receipt of the signal at the receiver 230.

In this particular case the receiver 230 is represented as a photo resistor; however, it is easily understood by those knowledgeable in the art that any electronic receiver that produces an electrical signal proportional to the strength of the light received could be used if properly conditioned. The signal from the source 230 is conditioned using the conditioning circuit 240 and then measured using the measurement circuit 250, which has two modes of operation either continuous or peak depending on the setting of the mode switch 252 and uses the reset switch 254 to reset the value in peak mode.

The voltage observed, which in this case corresponds to a fluorescence level is then displayed using the display circuit 260, which is comprised of a analog to digital converter 262, and LCD driver 264, and an LCD display 268. Additionally, an optional feature now shown, is for measurement circuit 250 to provide a zero offset voltage as an input to display circuit 260.

It is understood by those knowledge in the art that while the circuitry shown includes discrete components, it could easily be implemented by a microprocessor or software based via input to a computing device. Having provided a basis for understanding the measurement of fluorescence, embodiments consistent with the present disclosure for measuring the bilirubin level will now be discussed.

In a stand-alone system the fluorescence level is read in a fluorescence analyzer. The fluorescence analyzer is comprised of a housing containing a battery or other energy source, a light source, a sample chamber, a receiver with accompanying electronic circuitry for processing, measuring and displaying the level of fluorescence as described in FIG. 2.

The Light Source can be of any type (incandescent, fluorescent, LED, emitting light in the spectrum of the Bio-Fluorescent complex. In this present case, the Bilirubin/UnaG complex absorbs light maximally at 498 nm (This can be expressed as the Molar Extinction Coefficient, E at the wavelength of 498 nanometer (ε 498=77,300 Mole/cm2). Therefore, a light source emitting light in the range of 450-500 nm (Blue light) is desirable. But with proper filtering, any light source that includes these wave lengths can be utilized

When a sampling vial is inserted in the sampling chamber, the light ideally turns on automatically but could also be switch activated. The light from the source passes through the sample chamber and into the sample, some of which is absorbed. When absorbed, the bio-fluorescent complex emits a bright green fluorescence at 527 nm. Consequently, the receiver and associated circuitry will filter and select light of the appropriate frequency range (in this case 520-530 nm) for analysis. Prior research has also determined that the absolute fluorescence quantum yield for the Bilirubin/UnaG complex was determined to be 0.51 and stable at pH 4-11. (The fluorescence quantum yield is defined as the ratio of the number of photons emitted to the number of photons absorbed.)

It is worth repeating that the techniques associated with measurement of fluorescence are well known in the art and the method presented within FIG. 2 was simply presented as a representative technique for the purposes of understanding. Additional techniques include the use of a light reader interface attachable to a smart phone as well as techniques for analyzing fluorescence levels from a picture take remotely. In these techniques a known sample also represented in the picture is compared to the an object that is supposed to be fluorescing. For example, ImageJ is a public domain software that can be used for post processing of the image to determine the level of fluorescence. ImageJ is Java-based image processing program developed at the National Institutes of Health, which can be run as an online applet, a downloadable application, or on any computer with a Java 5 or later virtual machine. Downloadable distributions are available for Microsoft Windows, Mac OS, Mac OS X, Linux, and the Sharp Zaurus PDA. The source code for ImageJ is freely available.

Having described the underlying technology some representative implementations will now be presented.

Testing the blood serum bilirubin level can also be done with the bilirubin serum monitor similar in function to an electronic hand-held blood glucose monitoring device. In fact, early blood glucose monitors originally worked by detecting refracted light from a whole blood sample (e.g. U.S. Pat. No. 6,518,034 B1, Phillips et al., which is hereby incorporated by reference) and is anticipated that simply by adding appropriate filtering and/or replacing the light source many of the older devices could be easily and inexpensively converted for use as a bilirubin serum monitor.

During use, a sterile lance is used to puncture the fingertip (or other body part) and the resulting drop of blood is drawn via capillary action into a test strip (or placed into a well) connected to the reaction chamber of the bilirubin serum monitoring device. The reaction chamber (or well) contains a pre-determined concentration of the reagent protein buffered in the reagent solution and then the measurement is taken as described with reference to FIG. 2.

Additionally, it is anticipated that bilirubin serum monitor will have the ability to collect and store the data in a local or remote database incorporating one or more of either a patient ID or a unique strip ID. The data collected can also be transmitted via electronic messaging to a designated recipient such as the personal doctor or a remote monitoring application for follow-up.

The protocol for testing the blood serum bilirubin level using the bilirubin serum monitor comprises: using a sterile lance to puncture a clean fingertip; drawing a blood sample using a new test strip inserting; letting the test strip sit for at least 6-10 minutes or other pre-determined time increment in order to allow equilibrium (or near equilibrium such that there is no discernible difference based upon the measurement system accuracy) to be achieved, which may be shortened with the use of an accelerating factor or accelerating process such as heating (or cooling),and not more than 1 hour or other predetermined time increment (e.g. for instance after approximately 2 hours a form of oxidation, azo-pigmentation, can transform bilirubin from un-conjugated to conjugated) associated with stability of the reaction, which may be lengthened through the use of a stabilizing factor or stabilizing process such as cooling (or heating); measuring the fluorescence, which may be a peak fluorescence measurement, in the sample as a means of determining the bilirubin level and repeating the test at a pre-determined interval (e.g. every 4-6 hours); and comparing the results to determine if the bilirubin level has increased above a pre-determined threshold and if so issuing a warning that the patient may have an Ebola infection or other potential process associated with a spike in bilirubin levels.

In other embodiments, an ingested (or injectable) reagent proteins supplement solution can be used with potentially infected patients. The reagent proteins can be taken as a pill (food supplement) or administered by injection or intra-venously. The reagent proteins will form the bioluminescent complex when exposed to the bilirubin in the boby. The excreted bioluminescent compound can be visualized by exposure to the special UV light, thereby identifying sites contaminated by the infected human body fluids. The excreta and even the patient themselves can also be analyzed in a photo-spectrometer.

In still other embodiments, a cleaning solution containing the reagent proteins can used for the detection and identification of sites where any human body fluid and excreta (vomit, urine, fecal matter) are present. The method employed is as follows: a liquid or aerosol spray solution containing the protein marker is sprayed or applied on the surface of to be analyzed. If human waste (excreta) is present, the protein marker will form the bioluminescent complex when exposed to the bilirubin in the waste material. The resultant bioluminescent compound can be visualized by exposure to the special UV light, thereby confirming the presence of human body fluids on the surface. Note: prolonged exposure to UV light has also been shown to kill the Ebola virus; therefore, the same light used to detect potentially infected bodily fluids, through additional protocols can also be used to kill any actual virus.

Finally, it is to be understood that various different variants of the invention, including representative embodiments and extensions have been presented to assist in understanding the invention. It should be understood that such implementations are not to be considered limitations on either the invention or equivalents except to the extent they are expressly in the claims. It should therefore be understood that, for the convenience of the reader, the above description has only focused on a representative sample of all possible embodiments, a sample that teaches the principles of the invention. The description has not attempted to exhaustively enumerate all possible permutations, combinations or variations of the invention, since others will necessarily arise out of combining aspects of different variants described herein to form new variants, through the use of particular hardware or software, or through specific types of applications in which the invention can be used. That alternate embodiments may not have been presented for a specific portion of the description, or that further undescribed alternate or variant embodiments may be available for a portion of the invention, is not to be considered a disclaimer of those alternate or variant embodiments to the extent they also incorporate the minimum essential aspects of the invention, as claimed in the appended claims, or an equivalent thereof.

1. An Ebola detection method comprising:
adding a protein to a first bodily fluid, wherein the protein binds with bilirubin in first bodily fluid and in the bound state the protein is fluorescent;
measuring the fluorescence in order to determine the level of bilirubin in the first bodily fluid; and
determining based upon the relative level bilirubin in the first bodily fluid whether or not the first bodily fluids show indications of possible Ebola infection.

2. The method of claim 1 wherein the protein is UnaG.

3. The method of claim 1 further comprising:
adding a protein to a second bodily fluid, wherein the protein binds with bilirubin in second bodily fluid and in the bound state the protein is florescent and
measuring the fluorescence in order to determine the level of bilirubin in the second bodily fluid; and
wherein the relative level is based upon comparing the level bilirubin in the first bodily fluid with the level bilirubin in the second bodily fluid.

4. The method of claim 1 wherein the measuring comprises transmitting light in the range of 450-500 nm into the sample.

5. The method of claim 1 wherein the measuring comprises receiving light in the range of 520-530 nm emitted from the sample.

6. The method of claim 1 wherein the measuring is done by processing of a photograph using a known sample also represented in the picture.

7-17. (canceled)

18. A method of early detection of viral infections involving the breakdown of red blood cells comprising:
adding a protein to a first bodily fluid, wherein the protein binds with bilirubin in first bodily fluid and in the bound state the protein is fluorescent;
measuring the fluorescence in order to determine the level of bilirubin in the first bodily fluid; and
determining based upon the relative level bilirubin in the first bodily fluid whether or not the first bodily fluids show indications of possible viral infection.

19. The system of claim 18 wherein the viral infection is Ebola.

20. The method of claim 18 wherein the protein is UnaG.

21. The method of claim 18 further comprising:
adding a protein to a second bodily fluid, wherein the protein binds with bilirubin in second bodily fluid and in the bound state the protein is florescent and
measuring the fluorescence in order to determine the level of bilirubin in the second bodily fluid; and
wherein the relative level is based upon comparing the level bilirubin in the first bodily fluid with the level bilirubin in the second bodily fluid.

22. The method of claim 1 wherein the measuring comprises transmitting light in the range of 450-500 nm into the sample.

23. The method of claim 1 wherein the measuring comprises receiving light in the range of 520-530 nm emitted from the sample.

24. The method of claim 1 wherein the measuring is done by processing of a photograph using a known sample also represented in the picture.
